(19) United States
(12) Patent Application Publication
(10) Pub. No.: US 2026/0000279 A1
(43) Pub. Date: Jan. 1, 2026

(54) SYSTEMS, DEVICES, AND RELATED METHODS FOR STABLIZING MEDICAL DEVICES

(71) Applicant: Boston Scientific Scimed, Inc., Maple Grove, MN (US)

(72) Inventors: Colby HARRIS, Norfolk, MA (US); Ryan V. WALES, Shrewsbury, MA (US); Sean Powell, Holden, MA (US); Austin Grant JOHNSON, Worcester, MA (US)

(73) Assignee: Boston Scientific Scimed, Inc., Maple Grove, MN (US)

(21) Appl. No.: 19/250,937

(22) Filed: Jun. 26, 2025

Related U.S. Application Data
(60) Provisional application No. 63/664,963, filed on Jun. 27, 2024.

(51) Int. Cl. A61B 1/00; A61B 1/267; A61B 1/273; A61M 25/02

(52) U.S. Cl. CPC A61B 1/00154; A61B 1/2676; A61B 1/273; A61M 2025/0206; A61M 2025/022

(57) ABSTRACT

A medical device comprises: a base configured to be coupled to a solid structure or a bite block and one or more stabilizer assemblies. A first stabilizer assembly is configured to selectively inhibit a shaft of an endoscope from moving in an axial direction relative to the base or the bite block, and configured to allow a shaft of an endoscope to be rotated relative to the base or the bite block. A second stabilizer assembly is configured to selectively inhibit the shaft of the endoscope from rotating relative to the base or the bite block, and configured to allow the shaft of the endoscope to move in an axial direction relative to the base or the bite block.

### CROSS-REFERENCE TO RELATED APPLICATIONS

This application claims the benefit of priority to U.S. Provisional Application No. 63/664,963, filed on Jul. 27, 2024, which is incorporated by reference herein in its entirety.

### TECHNICAL FIELD

This disclosure relates generally to devices, systems, and methods for stabilizing a position of a medical device. More particularly, embodiments of the present disclosure relate to stabilizers for maintaining a position of a medical scope device during endoscopic procedures.

### BACKGROUND

Medical scope operators are performing more advanced procedures as their skill level increases and available technology advances. In some advanced procedures, physicians will get the scope device into a desired position, but may lose said desired position by the time an accessory device has been fed to the distal end of the endoscope. The inability to maintain or re-establish the desired position may be the most difficult aspect of many procedures. In an effort to stabilize a scope device during a procedure, physicians may resort to holding body positions that may be ergonomically challenging, or using boxes or platforms on a patient bed to precariously balance the scope device.

### SUMMARY OF THE DISCLOSURE

The disclosure includes systems, devices, and methods for stabilizing medical devices. The disclosed devices may also be used to stabilize a scope device during a procedure. For example, the disclosed devices may be incorporated into a base configured to be coupled to a solid structure or a bite block. Each of the aspects disclosed herein may include one or more of the features described in connection with any of the other disclosed aspects.

In an example, a medical device may comprise: (a) a base configured to be coupled to a solid structure or (b) a bite block; and an iris lock assembly coupled to the base or the bite block, the iris lock assembly comprising a plurality of leaves. The plurality of leaves may be configured to be actuated between an open position and a locked position.

Any of the aspects disclosed herein may include any of the following features, alone or in combination. In the locked position, the plurality of leaves may be configured to engage an outer surface of an endoscope. An interface may be configured to actuate the plurality of leaves. Rotation of the interface in a first direction may move the plurality of leaves radially inward, and rotation of the interface in a second direction, opposite the first direction, may move the plurality of leaves radially outward. The interface may include a texture, finish, or geometry to enable handling by a user. A bearing may be configured to allow the iris lock assembly to move in one of an axial direction or a rotational direction and to inhibit the iris lock assembly from moving in the other of the axial direction or the rotational direction. The bearing may include one or more rollers. The iris lock assembly may be a first iris lock assembly and the plurality of leaves may be a first plurality of leaves, and the medical device may further comprise a second iris lock assembly comprising a second plurality of leaves. The second plurality of leaves may be configured to be actuated between an open position and a locked position. The first iris lock assembly may further comprise a bearing configured to allow the first iris lock assembly to move in an axial direction relative to the base or the bite block and to inhibit the first iris lock assembly from rotating relative to the base or the bite block. The bearing may include one or more rollers. The one or more rollers may each have a rotational axis that is approximately perpendicular to a longitudinal axis of a central channel of the first iris lock assembly. The bearing may be a first bearing, and the second iris lock assembly may further comprise a second bearing configured to allow the second iris lock assembly to rotate relative to the base or the bite block and to inhibit the second iris lock assembly from moving in an axial direction relative to the base or the bite block. The second bearing may include one or more rollers, wherein the one or more rollers each have a rotational axis that is approximately parallel to a longitudinal axis of a central channel of the second iris lock assembly. In a configuration in which the first plurality of leaves is in a locked position and the second plurality of leaves is in an unlocked position, a shaft of an endoscope inserted through the central channels of the first iris lock assembly and the second iris lock assembly may be inhibited from rotating about a longitudinal axis of the shaft and may be allowed to move axially relative to the base or the bite block. In a configuration in which the first plurality of leaves is in an unlocked position and the second plurality of leaves is in a locked position, a shaft of an endoscope inserted through the central channels of the first iris lock assembly and the second iris lock assembly may be allowed to rotate about a longitudinal axis of the shaft and may be inhibited from moving axially relative to the base or the bite block.

In another aspect, a medical device may comprise: a base configured to be coupled to a solid structure or a bite block; a first stabilizer assembly configured to selectively inhibit a shaft of an endoscope from moving in an axial direction relative to the base or the bite block, and configured to allow a shaft of an endoscope to be rotated relative to the base or the bite block; and a second stabilizer assembly configured to selectively inhibit the shaft of the endoscope from rotating relative to the base or the bite block, and configured to allow the shaft of the endoscope to move in an axial direction relative to the base or the bite block.

Any of the aspects disclosed herein may include any of the following features, alone or in combination. In a configuration in which both the first stabilizer assembly and the second stabilizer assembly are locked, the shaft of the endoscope may be inhibited from moving in an axial direction and from rotating relative to the base or the bite block.

In another aspect, a medical device may comprise: a base configured to be coupled to a solid structure or a bite block; and a first iris lock assembly having a first channel. Actuation of the first iris lock assembly may be configured to transition the first iris lock assembly from a first configuration of the first iris lock assembly, in which the first channel has a first width, to a second configuration of the first iris lock assembly, in which the first channel has a second width, smaller than the first width. The medical device may further comprise: a second iris lock assembly having a second channel. Actuation of the second iris lock assembly may be configured to transition the second iris lock assembly from a first configuration of the second iris lock assembly, in which the second channel has a third width, to a second configuration of the second iris lock assembly, in which the second channel has a fourth width, smaller than the third width.

Any of the aspects disclosed herein may include any of the following features, alone or in combination. When the medical device has the first configuration of the first iris lock assembly and the second configuration of the second iris lock assembly, a shaft of a medical device inserted through the first channel and the second channel may be inhibited from rotating about a longitudinal axis of the shaft, and the shaft of the medical device may be allowed to move axially. When the medical device has the second configuration of the first iris lock assembly and the first configuration of the second iris lock assembly, a shaft of a medical device inserted through the first channel and the second channel may be allowed to rotate about a longitudinal axis of the shaft, and the shaft of the medical device may be inhibited from moving axially.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a perspective view of an exemplary medical device.

FIG. 2A depicts an iris lock of the exemplary medical device in an open position, according to one or more embodiments.

FIG. 2B depicts an iris lock of the exemplary medical device in a closed position, according to one or more embodiments.

FIG. 3 depicts an exploded view of an exemplary medical device, according to one or more embodiments.

FIG. 4 depicts another exemplary medical device, according to one or more embodiments.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

One of the largest challenges experienced by physicians during some procedures, such as endoluminal surgical procedures (ELS), is keeping an endoscope or other type of scope device (e.g., duodenoscope, endoscopic ultrasound (EUS) scope, or bronchoscope) stable during the procedure (e.g., the process of cutting and resecting tissue). As a physician performs the procedure, the endoscope may fall out of position, forcing the procedure (e.g., the resection) to stop and for the physician to spend time re-establishing position. This process of repositioning during a procedure may account for a large proportion of the time spent during the procedure (e.g., an ELS procedure).

Currently, to stabilize an endoscope, physicians may use overtubes, fill lumens with water, reposition the patient, put the endoscope down on a table to use multiple knobs and for stability, or resort to other measures that may not consistently work and/or provide limited stability. A device to stabilize the endoscope more effectively during tissue resection may significantly reduce the time of procedures while decreasing risks (e.g., of perforation), improving outcomes for patients and physicians alike.

Aspects of this disclosure seek to improve and ease a user's ability to stabilize a medical device, such as a medical scope (e.g., endoscope), during a medical procedure. Additionally, various aspects of this disclosure may help the user more efficiently perform endoscopic procedures, reduce overall procedure time, reduce overall procedure costs, etc.

One of ordinary skill in the art will appreciate that the systems, devices, and methods of this disclosure may be used with a variety of procedures and that the systems, devices, and methods of the disclosure may be applicable to various medical procedures beyond ELS.

Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or medical device may be used in connection with the devices and methods of this disclosure.

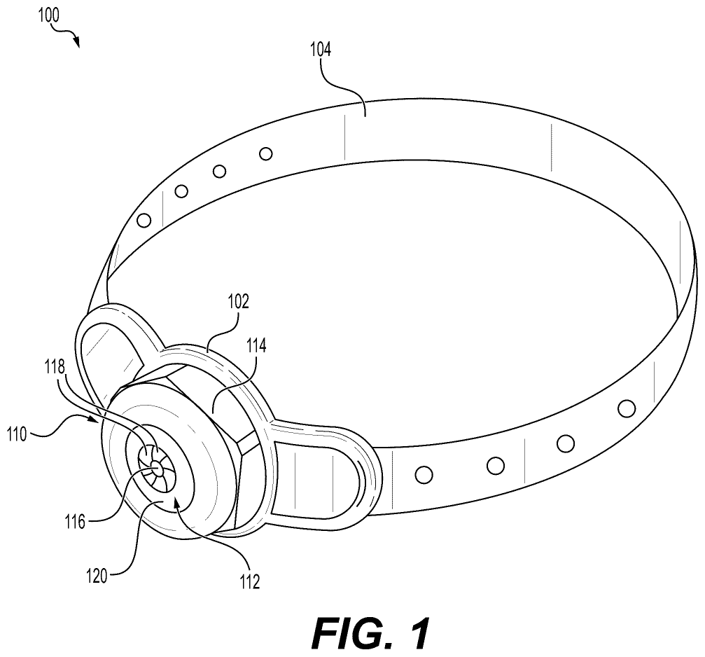

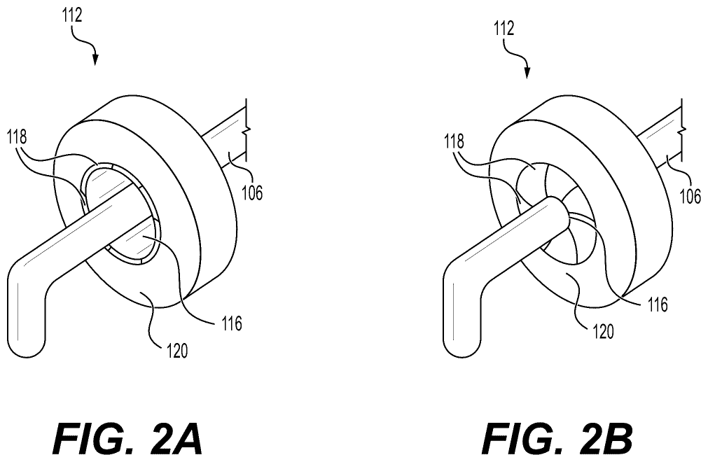

FIG. 1 depicts a perspective view of an exemplary medical device 100 including at least one stabilizer assembly 110. FIGS. 2A and 2B depict one or more components of a stabilizer assembly in an open and a locked position, respectively. Medical device 100 may include a bite block 102. During some endoscopic procedures, such as upper gastrointestinal (GI) procedures or bronchoscopy procedures, bite block may 102 be placed in the mouth of a patient to protect the patient's teeth and to protect delicate medical instruments that may be used in the procedures, such as an endoscope or other type of scope device.

Bite block 102 may be any suitable device used during a procedure to help ensure the patient's mouth remains open as well to protect an endoscope or other elongate medical device from damage. Bite block 102 may be placed in the patient's mouth and also secured around the patient's head using a strap 104. Bite block 102 may include a bite block channel (not shown but having any of the features of any bite block channel known in the art), through which an endoscope 106 (see FIGS. 2A and 2B) may be inserted during a procedure. The bite block channel may have a generally circular cross-section, although other suitable cross-sections also are contemplated.

Medical device 100 may further include a stabilizer assembly 110 for providing stability to endoscope 106 during a procedure. Stabilizer assembly 110 may be coupled to the bite block 102. Stabilizer assembly 110 may be configured to selectively prevent or limit endoscope 106 from being advanced, retracted, or rotated, while still allowing for articulation of a distal end of endoscope 106. Stabilizer assembly 110 may include at least one iris lock 112 and at least one interface 114 (e.g., a knob or the like) configured to be gripped by a user for actuating iris lock 112. Interface 114 may include a texture, finish, or geometry, such as a hexagonal shape as depicted in the example shown in FIG. 1, to enable handling by a user for optimal manipulation.

In the example, shown in FIG. 1, stabilizer assembly 110 of medical device 100 includes one iris lock 112. Iris lock 112 may comprise a lock channel 116, and a plurality of leaves 118 arranged circumferentially around lock channel 116. Lock channel 116 may be fluidly connected to the bite block channel and may also be configured to accept an endoscope 106 during a medical procedure. Lock channel 116 may have a generally circular cross-section, although other suitable cross-sections also are contemplated. Iris lock 112 further includes a generally cylindrical outer ring 120 arranged around lock channel 116.

A user, such as a physician, may actuate iris lock 112 via interface 114 (e.g., by rotation, by using a lever, etc.) to move leaves 118 radially inward and outward from outer ring 120 toward lock channel 116, adjusting the width of lock channel 116 to transition iris lock 112 between an open, or unlocked configuration, and a locked configuration. When an endoscope 106 inserted through lock channel 116 reaches a user's desired destination within the body lumen of a patient, the user may use interface 114 to move leaves 118 radially inward to decrease a width of lock channel 116 and to clamp onto or otherwise grip endoscope 106. Iris lock 112 may have any of the features of any iris lock, mechanical iris, or similar structure known in the art (e.g., gears, pivoting arms, etc.).

In some examples, interface 114 may be configured to rotate with respect to bite block 102. A rotation of interface 114 in a first direction (e.g. clockwise) may cause leaves 118 to move radially inward, locking an endoscope in place. In FIG. 2B, iris lock 112 is in a locked position, with leaves 118 of iris lock 112 moved radially inward until they are in a pressing engagement or otherwise in contact with a shaft 107 of endoscope 106. In the locked position, endoscope 106 may be locked in position, preventing or limiting endoscope 106 from being advanced, retracted or rotated, while still allowing for articulation of endoscope 106.

Conversely, a rotation of interface 114 in a second direction opposite the first direction (e.g., counter-clockwise) may cause leaves 118 to move radially outward, enlarging lock channel 116 allowing for free movement of an endoscope. In FIG. 2A, iris lock 12 is in an open, or unlocked, position, such that lock channel 116 is sized to be larger than an endoscope cross-sectional area, allowing shaft 107 of endoscope 106 to pass freely therethrough. Lock channel 116 may have a smaller width in the locked position (FIG. 2B) than in the open, or unlocked, position (FIG. 2A). In the open, or unlocked, position, leaves 118 of iris lock 112 are disposed radially outward relative to the locked position, enlarging interior channel 116. Leaves 118 may optionally be retracted into cover ring 120 in the open, or unlocked, position. In the open position, endoscope 106 passing through lock channel 116 may be freely advanced, retracted, rotated and articulated.

In another example, interface 114 may be mechanically connected to bite block 102 by a linkage (not shown) that allows for linear movement between interface 114 and bite block 102. Movement of interface 114 in a first direction (e.g., in a proximal direction) may cause leaves 118 to move radially inward, locking an endoscope in place. Conversely, movement of interface 114 in a second direction opposite the first direction (e.g., in a distal direction) may cause leaves 118 to move radially outward, enlarging lock channel 116 allowing for free movement of an endoscope.

In other examples, leaves 118 may be actuated by linear, pneumatic or hydraulic actuators known in the art. These actuators may be controlled by, for example, a foot pedal or wire. Leaves 118 may alternatively be actuated by other manual actuators, such as levers, buttons, sliders, etc.

The example described in FIG. 1 allows for two states: (1) free movement of an endoscope within a lock channel such that the endoscope may be freely advanced, retracted, rotated, and articulated, and (2) a locked position, preventing or limiting an endoscope from being advanced, retracted or rotated, while still allowing for articulation of endoscope.

Although stabilizer assembly 110 is shown as being associated with bite block 102, stabilizer assembly 110 may alternatively be associated with other structures, such as table-mounted structures, structures mounted on IV poles or the like, tabletop structures, or other patient-mounted structures.

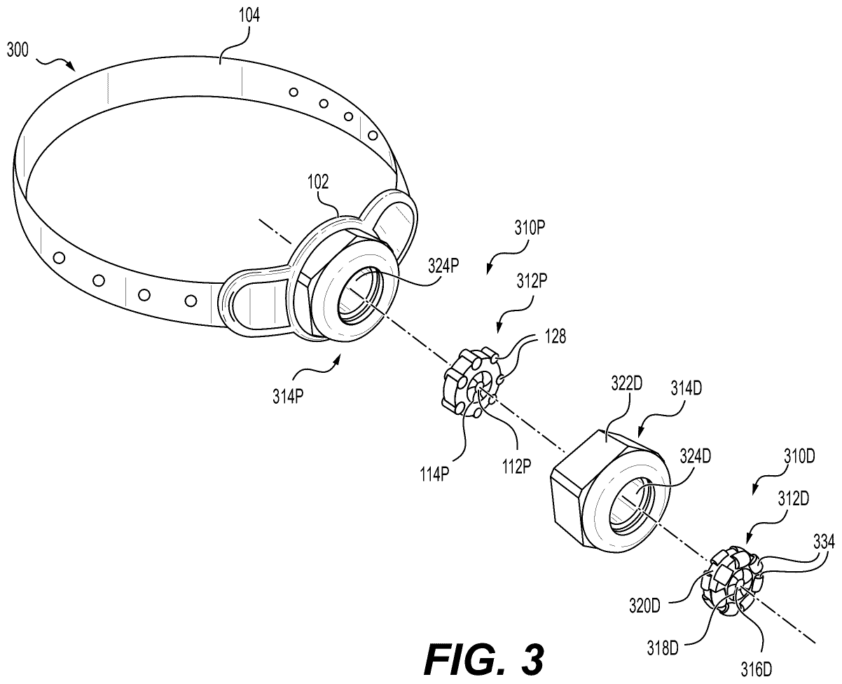

FIG. 3 depicts an exploded view of an exemplary medical device 300 that may include, among other elements: a first stabilizer assembly 310D comprising a first iris lock 312D, a first interface 314D, and a first bearing 330D positioned between first iris lock 312D and first interface 314D, first bearing 330D including a plurality of radial rollers 332. The medical device 300 may also include a second stabilizer assembly 310P comprising a second iris lock 312P, a second interface 314P, and a second bearing 330P positioned between second iris lock 312P and second interface 314P, second bearing 330P including a plurality of linear rollers 334. Medical device 300 includes substantial similarities to medical device 100 described above and may have any of the properties of medical device 100 as described in FIGS. 1-2B. Stabilizer assemblies 310D and 310P may have any of the features of stabilizer assembly 110.

Medical device 300 may include a bite block 302 coupled to stabilizer assemblies 310D and 310P. Bite block 302 may be any suitable device used during a procedure to help ensure the patient's mouth remains open as well to protect an endoscope or other elongate medical device from damage. Bite block 302 may be placed in the patient's mouth and also secured around the patient's head using a strap 304. Bite block 302 may include a bite block channel (not shown), through which an endoscope 106 (see FIGS. 2A and 2B) may be inserted during a procedure. The bite block channel may have a generally circular cross-section, although other suitable cross-sections also are contemplated.

In the example shown in FIG. 3, medical device 300 may allow for up to four states: (1) a free state, providing free movement of an endoscope such that the endoscope may be freely advanced, retracted, rotated and articulated; (2) a locked state, preventing or limiting an endoscope from being advanced, retracted or rotated, while still allowing for articulation of endoscope via bending of an articulation joint of the endoscope; (3) a linearly locked state, preventing or limiting an endoscope from being advanced or retracted, while still allowing the endoscope to be freely rotated and articulated via bending of an articulation joint of the endoscope; and (4) a rotationally locked state, preventing or limiting an endoscope from being rotated, while still allowing the endoscope to be freely advanced, retracted, and articulated via bending of an articulation joint of the endoscope.

The four states may be achieved by selectively actuating first iris lock 312D and/or second iris lock 312P to an open position or actuating first iris lock 312D and/or second iris lock 312P to a closed position. In particular: (1) the free state may be achieved by actuating both first iris lock 312D and second iris lock 312P into an open position; (2) the locked state may be achieved by actuating both first iris lock 312D and second iris lock 312P into a closed position; (3) the linearly locked state may be achieved by actuating first iris lock 312D to a closed position and actuating second iris lock 312P to an open position; and (4) the rotationally locked state may be achieved by actuating first iris lock 312D to an open position and second iris lock 312P to a closed position.

First interface 314D may be configured to be gripped by a user for actuating first iris lock 312P. First interface 314D may include an outer surface 322D with a texture, finish, or geometry, such as a hexagonal shape as depicted in the example shown in FIG. 3, to enable handling by a user for optimal manipulation. First interface 314D may further include an inner channel 324D configured to receive first iris lock 312P.

First iris lock 312D may be substantially similar to iris lock 112 described with respect to FIGS. 1-2B and may have any of the properties of iris lock 112. First iris lock 312D may comprise a first lock channel 316P, and a plurality of leaves 318D arranged circumferentially around first lock channel 316P. First lock channel 316D may be fluidly connected to the bite block channel and may also be configured to accept an endoscope 106 during a medical procedure. First lock channel 316D may have a generally circular cross-section, although other suitable cross-sections also are contemplated. First iris lock 312D may further include a generally cylindrical outer ring 320D arranged around first lock channel 316P.

The first bearing may allow for iris lock 312D to rotate relative to bite block 302 but may inhibit iris lock 312D from moving axially/longitudinally (i.e., in an axial or longitudinal direction) with respect to bite block 302. The axial or longitudinal direction is a direction that is approximately parallel to or coaxial with a central longitudinal axis of first lock channel 316D and/or a longitudinal axis of endoscope 106. The first bearing may include a plurality of radial rollers 332 (e.g., roller bearings) arranged on a radially outer circumference of first outer ring 320P. Radial rollers 332 of the first bearing may be configured to engage an interior surface of inner channel 324D of first interface 314P. Radial rollers 332 may allow for rotational movement of first iris lock 312D relative to first interface 314D and bite block 302. For example, an axis of rotation of each of radial rollers 332 may be approximately parallel to a central longitudinal axis of first lock channel 316P.

In some examples, first iris lock may be actuated via first interface 314P. First interface 314D may be configured to rotate with respect to bite block 302. A rotation of first interface 314D in a first direction (e.g. clockwise) may cause leaves 318D to move radially inward, locking an endoscope in place. In the locked position, endoscope 106 may be locked in position, preventing or limiting shaft 107 of endoscope 106 from being advanced, or retracted (moved axially or longitudinally) relative to first lock channel 316P, while still allowing for articulation of endoscope 106 via an articulation joint of endoscope 106 controlled with a handle of endoscope 106. Because first iris lock 312D is free to rotate relative to bite block 302, the locked position of iris lock 312D still allows for rotation of shaft 107 of endoscope 106 about a central longitudinal axis of the shaft of endoscope 106.

Conversely, a rotation of first interface 314D in a second direction opposite the first direction (e.g., counter-clockwise) may cause leaves 318D to move radially outward, enlarging first lock channel 316D allowing for free movement of endoscope 106 relative to first lock channel 316D when second iris lock 312P is not locked. In the open position of first iris lock 312D and second iris lock 312P, endoscope 106 passing through first lock channel 316P may be freely advanced, retracted, rotated and articulated.

Similarly, second interface 314P may be configured to be gripped by a user for actuating second iris lock 312D. Second interface 314P may include an outer surface 322P with a texture, finish, or geometry, such as a hexagonal shape as depicted in the example shown in FIG. 3, to enable handling by a user for optimal manipulation. Second interface 314P may further include an inner channel 324P configured to receive second iris lock 312D.

Second iris lock 312P may be substantially similar to iris lock 112 described with respect to FIGS. 1-2B and first iris lock 312D and may have any of the properties of iris lock 112 and first iris lock 312D, except as specified below. Second iris lock 312P may comprise a second lock channel 316P, and a plurality of leaves 318P arranged circumferentially around second lock channel 316P. Second lock channel 316P may be connected to the bite block channel and may also be configured to accept an endoscope 106 during a medical procedure. Second lock channel 316P may have a generally circular cross-section, although other suitable cross-sections also are contemplated. Second iris lock 312P may further include a generally cylindrical outer ring 320P arranged around second lock channel 316D.

The second bearing may allow second iris lock 312P to move axially/longitudinally with respect to bite block 302 but may inhibit iris lock 312P from rotating relative to bite block 302. The second bearing may include a plurality of linear rollers 334 arranged on a radially outer circumference of second outer ring 320D. Linear rollers 334 may be configured to engage an interior surface of inner channel 324P of interface 314P. Linear rollers 334 may allow for linear movement of second iris lock 312P relative to second interface 314P and bite block 302. For example, linear rollers 334 may have axes of rotation that extend tangentially to an outer surface of second outer ring 320D. The axes of rotation of linear rollers 334 may be approximately perpendicular to a central longitudinal axis of second lock channel 316D.

In some examples, second iris lock may be actuated via second interface 314D. Second interface 314P may be configured to rotate with respect to bite block 302. A rotation of second interface 314P in a first direction (e.g. clockwise) may cause leaves 318P to move radially inward, locking an endoscope in place. In the locked position, shaft 107 of endoscope 106 may be prevented from being rotated about its longitudinal axis relative to second lock channel 316D, while still allowing for articulation of endoscope 106 via an articulation joint of endoscope 106 controlled by a handle of endoscope 106. Because second iris lock 312P is free to move linearly (axially or longitudinally) relative to bite block 302, the locked position of iris lock 312P also allows for advancement and retraction axially or longitudinally of shaft 107 of endoscope 106.

Conversely, a rotation of second interface 314P in a second direction opposite the first direction (e.g., counter-clockwise) may cause leaves 318 to move radially outward, enlarging second lock channel 316P allowing for free movement of endoscope 106 relative to second lock channel 316P when first iris lock 312D is not locked. In the open position of first iris lock 312D and second iris lock 312P, endoscope 106 passing through second lock channel 316P may be freely advanced, retracted, rotated and articulated.

In the example shown in FIG. 3, first stabilizer assembly 310D is disposed more distally than second stabilizer assembly 310P in relation to bite block 302. However, the arrangement of the first stabilizer assembly 310D and second stabilizer assembly 310P may be reversed such that first stabilizer assembly 310D is arranged more proximally than second stabilizer assembly 310P.

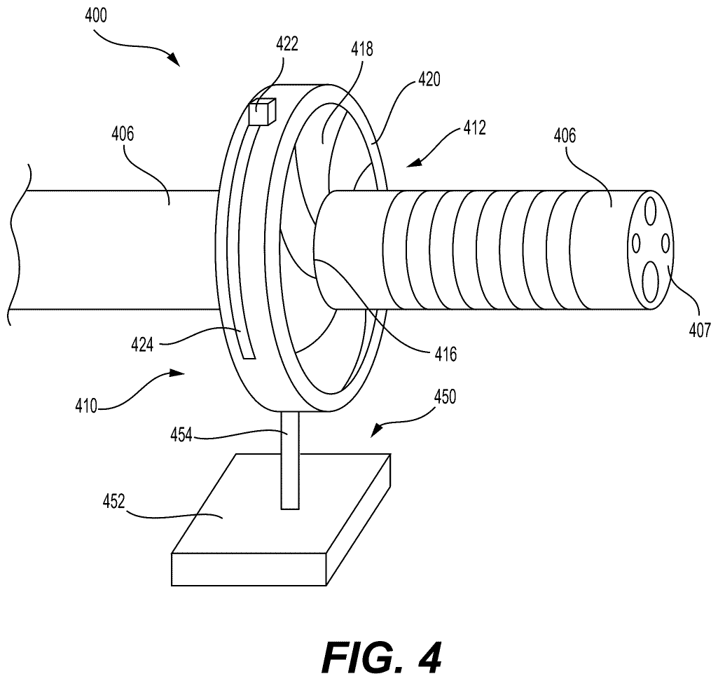

FIG. 4 depicts a perspective view of another exemplary medical device 400 including at least one stabilizer assembly 410. Medical device 400 may be substantially similar to medical device 100 or medical device 300 described in FIGS. 1-3, except as described below. Medical devices 100 or 300 may have any of the properties of medical device 400.

Medical device 400 may include a base 450 for placing or mounting stabilizer assembly 410 to, for example, a table or other stationary or stable object (e.g., a bed or a platform). Base 450 may include a base plate 452 and a base arm 454 for attaching to stabilizer assembly 410. Base plate 452 may include, for example, suction cups, adhesive material, or other mounting components for stabilizing medical device 400 on a table or other generally stationary or stable object. For example, base plate 452 may be coupled to any type of clamp device known in the art for coupling base plate 452 to the table or other surface.

The at least one stabilizer assembly 410 may be substantially similar to stabilizer assembly 110 described in FIGS. 1-2, or either of first stabilizer assembly 310P or second stabilizer assembly 310P described in FIG. 3. In other examples, medical device 400 may include multiple stabilizer assemblies, such as one substantially similar to first stabilizer assembly 310P and one substantially similar to second stabilizer assembly 310P.

As such, stabilizer assembly 410 may be configured to selectively prevent or limit an endoscope 406 from being advanced, retracted, or rotated, while still allowing for articulation of a distal end 407 of endoscope 406. For example, stabilizer assembly 410 may include at least one iris lock 412.

Iris lock 412 may comprise a lock channel 416, and a plurality of leaves 418 arranged circumferentially around lock channel 416. Lock channel 416 may be configured to accept endoscope 406 during a medical procedure. Iris lock 412 further includes a generally cylindrical outer ring 420 arranged around lock channel 416. In aspects, radially inner portions of leaves 418 may include flexible materials (e.g., rubber, silicone, or polymer) so that leaves 418 are better secured within channel 416, as described below. Alternatively, radially inner portions of leaves 418 may be coupled to an O-ring or similar element.

In some examples, a user, such as a physician, may actuate iris lock 412 via any actuating element described in FIGS. 1-3. In other examples, a user may actuate iris lock 412 via a sliding tab 422 arranged on outer ring 420. Sliding tab 422 may be arranged in a groove 424 in outer ring 420. Sliding tab 422 may be connected via gears, linkages, or other elements to leaves 418. A user may actuate iris lock 412 via sliding tab 422 to move leaves 418 radially inward and outward from outer ring 420 toward and away from lock channel 416, adjusting the width of lock channel 416 to transition iris lock 412 between an open, or unlocked configuration, and a locked configuration.

An actuation of sliding tab 422 in a first direction (e.g. clockwise) may cause leaves 418 to move radially inward, locking endoscope 406 in place. Conversely, an actuation of sliding tab 422 in a second direction opposite the first direction (e.g., counter-clockwise) may cause leaves 418 to move radially outward, enlarging lock channel 116 allowing for free movement of endoscope 406.

In other aspects, stabilizer assembly 410 may include a Touhy Borst structure, in alternative to leaves 418. Stabilizer assembly 410 may have any feature of any Touhy Borst structure known in the art. For example, stabilizer assembly 410 may include an O-ring, a flexible (e.g., silicone or other material) sleeve, a body and a cap. Rotation of the cap relative to the body (or vice versa) may cause the sleeve to be squeezed around endoscope 406. Alternatively, similar other structures may be used that produce a radially inward force on endoscope 406 to secure endoscope 406 within stabilizer assembly 410.

Other embodiments of the present disclosure will be apparent to those skilled in the art from consideration of the specification and practice of the embodiments disclosed herein. It is intended that the specification and examples be considered as exemplary only, with a true scope and spirit of the present disclosure being indicated by the following claims.

We claim:

1. A medical device comprising:
(a) a base configured to be coupled to a solid structure or (b) a bite block; and
an iris lock assembly coupled to the base or the bite block, the iris lock assembly comprising a plurality of leaves, wherein the plurality of leaves is configured to be actuated between an open position and a locked position.

2. The medical device of claim 1, wherein, in the locked position, the plurality of leaves is configured to engage an outer surface of an endoscope.

3. The medical device of claim 1, further comprising an interface configured to actuate the plurality of leaves.

4. The medical device of claim 3, wherein rotation of the interface in a first direction moves the plurality of leaves radially inward, and rotation of the interface in a second direction, opposite the first direction, moves the plurality of leaves radially outward.

5. The medical device of claim 3, wherein the interface includes a texture, finish, or geometry to enable handling by a user.

6. The medical device of claim 1, wherein:
the iris lock assembly further comprises a bearing configured to allow the iris lock assembly to move in one of an axial direction or a rotational direction and
to inhibit the iris lock assembly from moving in the other of the axial direction or the rotational direction.

7. The medical device of claim 6, wherein the bearing includes one or more rollers.

8. The medical device of claim 1, wherein the iris lock assembly is a first iris lock assembly, and the plurality of leaves is a first plurality of leaves, the medical device further comprising:
a second iris lock assembly comprising a second plurality of leaves, wherein the second plurality of leaves is configured to be actuated between an open position and a locked position.

9. The medical device of claim 8, wherein:
the first iris lock assembly further comprises a bearing configured to allow the first iris lock assembly to move in an axial direction relative to the base or the bite block and to inhibit the first iris lock assembly from rotating relative to the base or the bite block.

10. The medical device of claim 9, wherein the bearing includes one or more rollers.

11. The medical device of claim 10, wherein the one or more rollers each have a rotational axis that is approximately perpendicular to a longitudinal axis of a central channel of the first iris lock assembly.

12. The medical device of claim 9, wherein the bearing is a first bearing, and wherein the second iris lock assembly further comprises a second bearing configured to allow the second iris lock assembly to rotate relative to the base or the bite block and to inhibit the second iris lock assembly from moving in an axial direction relative to the base or the bite block.

13. The medical device of claim 12, wherein the second bearing includes one or more rollers, wherein the one or more rollers each have a rotational axis that is approximately parallel to a longitudinal axis of a central channel of the second iris lock assembly.

14. The medical device of claim 13, wherein, in a configuration in which the first plurality of leaves is in a locked position and the second plurality of leaves is in an unlocked position, a shaft of an endoscope inserted through the central channels of the first iris lock assembly and the second iris lock assembly is inhibited from rotating about a longitudinal axis of the shaft and is allowed to move axially relative to the base or the bite block.

15. The medical device of claim 13, wherein, in a configuration in which the first plurality of leaves is in an unlocked position and the second plurality of leaves is in a locked position, a shaft of an endoscope inserted through the central channels of the first iris lock assembly and the second iris lock assembly is allowed to rotate about a longitudinal axis of the shaft and is inhibited from moving axially relative to the bite block.

16. A medical device comprising:
(a) a base configured to be coupled to a solid structure or (b) a bite block;
a first stabilizer assembly configured to selectively inhibit a shaft of an endoscope from moving in an axial direction relative to the base or the bite block, and configured to allow a shaft of an endoscope to be rotated relative to the base or the bite block; and
a second stabilizer assembly configured to selectively inhibit the shaft of the endoscope from rotating relative to the base or the bite block, and configured to allow the shaft of the endoscope to move in an axial direction relative to the base or the bite block.

17. The medical device of claim 16, wherein, in a configuration in which both the first stabilizer assembly and the second stabilizer assembly are locked, the shaft of the endoscope is inhibited from moving in an axial direction and from rotating relative to the base or the bite block.

18. A medical device comprising:
(a) a base configured to be coupled to a solid structure or (b) a bite block;
a first iris lock assembly having a first channel, wherein actuation of the first iris lock assembly is configured to transition the first iris lock assembly from a first configuration of the first iris lock assembly, in which the first channel has a first width, to a second configuration of the first iris lock assembly, in which the first channel has a second width, smaller than the first width; and
a second iris lock assembly having a second channel, wherein actuation of the second iris lock assembly is configured to transition the second iris lock assembly from a first configuration of the second iris lock assembly, in which the second channel has a third width, to a second configuration of the second iris lock assembly, in which the second channel has a fourth width, smaller than the third width.

19. The medical device of claim 18, wherein, when the medical device has the first configuration of the first iris lock assembly and the second configuration of the second iris lock assembly, a shaft of a medical device inserted through the first channel and the second channel is inhibited from rotating about a longitudinal axis of the shaft, and the shaft of the medical device is allowed to move axially.

20. The medical device of claim 18, wherein, when the medical device has the second configuration of the first iris lock assembly and the first configuration of the second iris lock assembly, a shaft of a medical device inserted through the first channel and the second channel is allowed to rotate about a longitudinal axis of the shaft, and the shaft of the medical device is inhibited from moving axially.
